(19) United States
(12) Patent Application Publication
(10) Pub. No.: US 2026/0058028 A1
(43) Pub. Date: Feb. 26, 2026

(54) DIAGNOSIS SUPPORT DEVICE, DIAGNOSIS SUPPORT METHOD, AND RECORDING MEDIUM

(71) Applicant: NEC Corporation, Tokyo (JP)

(72) Inventor: Atsushi MARUGAME, Tokyo (JP)

(73) Assignee: NEC Corporation, Tokyo (JP)

(21) Appl. No.: 19/303,596

(22) Filed: Aug. 19, 2025

(30) Foreign Application Priority Data
Aug. 26, 2024 (JP) 2024-144144

(51) Int. Cl. G16H 80/00; G16H 15/00; G16H 40/20

(52) U.S. Cl. CPC G16H 80/00; G16H 15/00; G16H 40/20

(57) ABSTRACT

In order to provide a diagnosis support device that creates a draft medical certificate and sends the draft medical certificate to an appropriate department, in a diagnosis support device, a prompt creation means acquires diagnosis information and creates a prompt including the diagnosis information. An estimation means inputs the prompt into a diagnosis support AI and estimates case candidates. A draft creation means creates a draft of a medical certificate based on the case candidates. A selection means selects a destination for sending the draft of the medical certificate based on the case candidates. Thus, it is possible for the diagnosis support device to support a doctor in decision-making in the field of healthcare.

### INCORPORATION BY REFERENCE

This application is based upon and claims the benefit of priority from Japanese Patent Application 2024-144144, filed on August 26, 2024, the disclosure of which is incorporated herein in its entirety by reference.

### TECHNICAL FIELD

The present disclosure relates to technology for Supporting diagnosis.

### BACKGROUND ART

Systems for supporting diagnosis using AI (Artificial Intelligence) are known. For example, Patent Document 1 discloses a method of generating diagnostic information from patient interview information using generative AI.

Patent Document 1: Japanese Patent No. 7450310

### SUMMARY

The diagnostic information in Patent Document 1 is used as an aid for a physician's diagnosis. However, with the method of Patent Document 1, the diagnostic information may not always be effectively utilized, for example, when it includes information outside the physician's specialty.

One object of the present disclosure is to provide a diagnosis support device that creates a draft of a medical certificate and sends the draft of the medical certificate to an appropriate department.

According to an example aspect of the present invention, there is provided a diagnosis support device including:

at least one memory configured to store instructions; and

at least one processor configured to execute the instructions to:

acquire diagnosis information and creates a prompt including the diagnosis information;

input the prompt into a diagnosis support AI and estimates case candidates;

create a draft of a medical certificate based on the case candidates; and

select a destination for sending the draft of the medical certificate based on the case candidates.

According to another example aspect of the present invention, there is provided a diagnosis support method executed by a computer, including:

acquiring diagnosis information and creating a prompt including the diagnosis information;

inputting the prompt into a diagnosis support AI and estimating case candidates;

creating a draft of a medical certificate based on the case candidates; and

selecting a destination for sending the draft of the medical certificate based on the case candidates.

According to still another example aspect of the present invention, there is provided a non-transitory computer-readable recording medium storing a program causing a computer to execute processing of:

acquiring diagnosis information and creating a prompt including the diagnosis information;

inputting the prompt into a diagnosis support AI and estimating case candidates;

creating a draft of a medical certificate based on the case candidates; and

selecting a destination for sending the draft of the medical certificate based on the case candidates.

EFFECT

According to the present disclosure, it is possible to provide a diagnosis support device that creates a draft medical certificate and sends the draft medical certificate to an appropriate department.

### BRIEF DESCRIPTION OF THE DRAWINGS

FIG. 1 illustrates an overall configuration of a diagnosis support system to which a diagnosis support device according to the present disclosure is applied.

FIG. 2 is a block diagram illustrating a hardware configuration of the diagnosis support device according to the present disclosure.

FIG. 3 is a block diagram illustrating a functional configuration of the diagnosis support device according to the present disclosure.

FIG. 4 is an example of a draft medical certificate.

FIG. 5 is a flowchart of processing by the diagnosis support device according to the present disclosure.

FIG. 6 is a block diagram illustrating the functional configuration of another diagnosis support device according to the present disclosure.

FIG. 7 is a flowchart of processing by another diagnosis support device according to the present disclosure.

FIG. 8 is a block diagram illustrating the functional configuration of another diagnosis support device according to the present disclosure.

FIG. 9 is a flowchart of processing by another diagnosis support device according to the present disclosure.

### EXAMPLE EMBODIMENTS

Preferred example embodiments of the present disclosure will be described with reference to the accompanying drawings.

<First Example Embodiment>

Medical institutions may include a plurality of departments and divisions (hereinafter also referred to as "medical departments"). In examining a patient, there are cases where the case is minor for the medical department that examined the patient, but not minor for other medical departments, and for example, information regarding that case may be stored in the medical institution's hospital information system (HIS: Hospital Information System). However, currently, it takes time to link such information.

Therefore, although details will be described later, the diagnosis support device of this example embodiment uses generative AI to estimate a case from information obtained during the examination of the patient. Then, the diagnosis support device creates a draft of a medical certificate for the estimated case and sends the draft of the medical certificate to an appropriate medical department. In this way, the diagnosis support device of this example embodiment can quickly estimate cases across medical departments and create draft diagnoses.

[Overall Configuration]

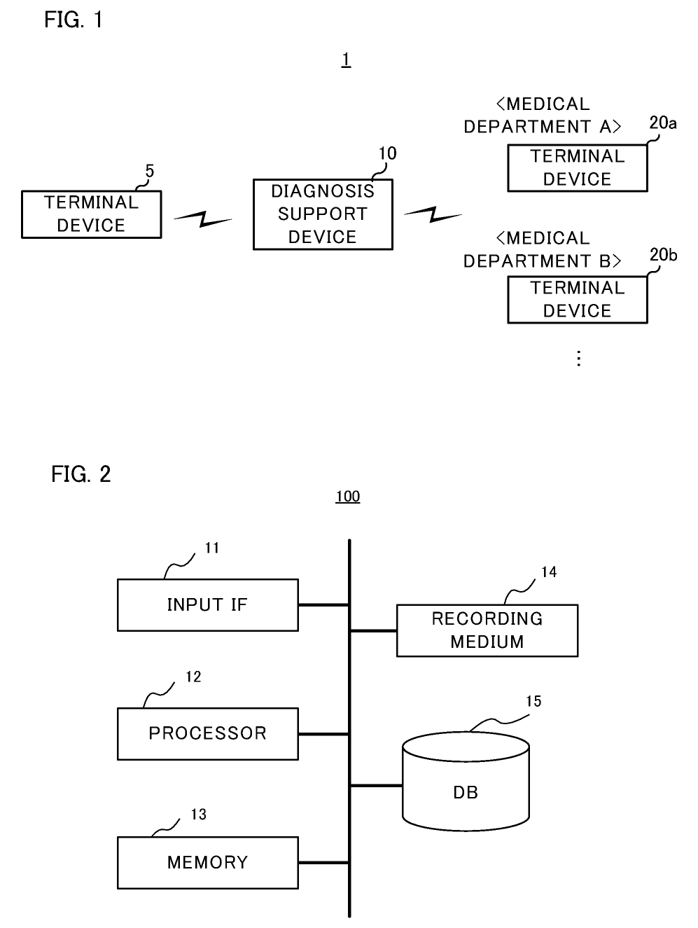

FIG. 1 illustrates the overall configuration of a diagnosis support system to which the diagnosis support device according to the present disclosure is applied. The diagnosis support system 1 includes a terminal device 5, a diagnosis support device 10, and terminal devices 20 of the plurality of medical departments. Note that with regard to the terminal device 20 used in medical departments, a subscript is added to distinguish between individual departments, whereas when no distinction is necessary, it is simply referred to as "terminal device 20".

The terminal device 5 is operated by a doctor or the like and sends information obtained during the examination of the patient to the diagnosis support device 10. The information obtained during the examination of the patient includes, for example, conversations between the doctor and the patient, and medical images taken during the examination. The information obtained during the examination of the patient is hereinafter also referred to as "diagnosis time information." The terminal device 5 is formed, for example, by a personal computer, and communicates with the diagnosis support device 10 via a network such as the Internet.

The diagnosis support device 10 estimates a patient's case from the diagnosis time information and creates the draft of the medical certificate. Then, the diagnosis support device 10 sends the draft of the medical certificate to an appropriate medical department. The diagnosis support device 10 is configured, for example, by a server device and communicates with the terminal device 5 and the terminal device 20 via the network such as the Internet.

The terminal device 20 of the medical department is operated by a person in charge of the medical department or the like, and is for viewing the draft of the medical certificate received from the diagnosis support device 10. The terminal device 20 is configured, for example, by a personal computer or the like, and communicates with the diagnosis support device 10 via the network such as the Internet.

[Hardware Configuration]

FIG. 2 is a block diagram illustrating the hardware configuration of the diagnosis support device 10 according to the first example embodiment. As illustrated, the diagnosis support device 10 includes an interface (I/F) 11, a processor 12, a memory 13, a recording medium 14, and a database (DB) 15.

The I/F 11 communicates with the terminal device 5 and the terminal device 20 via the network such as the Internet.

The processor 12 is a computer such as a CPU (Central Processing Unit), and controls the entire diagnosis support device 10 by executing a program prepared in advance. Note that the processor 12 may be a GPU (Graphics Processing Unit), DSP (Digital Signal Processor), MPU (Micro Processing Unit), FPU (Floating Point number Processing Unit), PPU (Physics Processing Unit), TPU (Tensor Processing Unit), quantum processor, microcontroller, or a combination thereof. The processor 12 executes the draft creation process described later.

The memory 13 is configured by ROM (Read Only Memory), RAM (Random Access Memory), and the like. The memory 13 is also used as a working memory during the execution of various processes by the processor 12.

The recording medium 14 is a non-volatile and non-transitory recording medium such as a disk-shaped recording medium or a semiconductor memory, and is formed to be attachable to and detachable from the diagnosis support device 10. The recording medium 14 records various programs executed by the processor 12. In a case where the diagnosis support device 10 executes various processes, the programs recorded in the recording medium 14 are loaded into the memory 13 and executed by the processor 12.

The DB 15 stores, for example, a table describing rules associating cases and medical departments, which will be described later.

In addition to the above, the diagnosis support device 10 may include a display device such as a liquid crystal display, and an input device such as a keyboard or a mouse. These display devices and input devices are used, for example, by the administrator of the diagnosis support device 10 to perform necessary management.

[Functional Configuration]

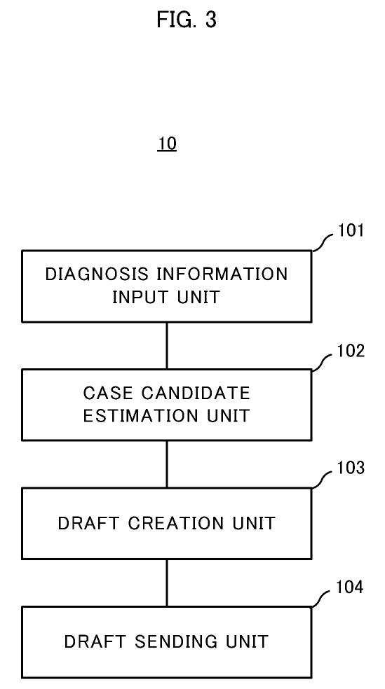

FIG. 3 is a block diagram illustrating the functional configuration of the diagnosis support device 10 of the first example embodiment. The diagnosis support device 10 functionally includes a diagnosis information input unit 101, a case candidate estimation unit 102, a draft creation unit 103, and a draft sending unit 104.

The diagnosis time information is input to the diagnosis support device 10 from the terminal device 5 via the I/F 11. The diagnosis time information includes audio data of conversations between the doctor and the patient, medical image data taken during examination, and the like. The diagnosis time information is input to the diagnosis information input unit 101.

The diagnosis information input unit 101 creates a prompt to be input to the generative AI, and outputs the prompt to the case candidate estimation unit 102. For example, the diagnosis information input unit 101 attaches diagnosis time information to a prompt as illustrated below, and outputs the prompt to the case candidate estimation unit 102. Note that the diagnosis information input unit 101 may attach the audio data directly to the prompt, or may convert the audio data into text data using an existing voice recognition model and insert the converted text data into the prompt.

(Example of prompt) The attached data is diagnosis time information. Please output the possible cases from the attached data. Also, please tell me the basis for selecting that case.

The case candidate estimation unit 102 estimates candidates of cases (hereinafter also referred to as "case candidates") using a generative AI prepared in advance. The generative AI used by the case candidate estimation unit 102 is hereinafter referred to as a "diagnosis support AI." The diagnosis support AI takes the prompt as input, and outputs a case and the basis for selecting that case. In addition to the case and the basis for selecting that case, the diagnosis support AI may output the likelihood of the case (hereinafter also referred to as "case certainty"). The certainty of the case may be expressed in words such as "most likely," "second most likely," or may be expressed numerically. The case candidate estimation unit 102 outputs the output of the diagnosis support AI to the draft creation unit 103 as the case candidates.

The diagnosis support AI is created by additionally training general-purpose generative AI such as ChatGPT by OpenAI or Gemini by Google. Additional training includes fine-tuning and transfer learning. For example, the case candidate estimation unit 102 performs additional training of the general-purpose generative AI using a dataset of pairs of diagnosis time information and answers to the diagnosis time information. Accordingly, it is possible to create the diagnosis support AI specialized in the medical field. Note that the answer to the diagnosis time information is information concerning the case (hereinafter, also referred to as "case information"), and the case information stored in the HIS can be used.

Moreover, the case candidate estimation unit 102 may use RAG (Retrieval Augmented Generation) instead of the diagnosis support AI. RAG is a technology that combines the general-purpose generative AI and an external information source to generate a highly reliable answer. The case candidate estimation unit 102 can use a database owned by a medical institution or the like as an external information source.

The draft creation unit 103 creates the draft of the medical certificate based on the output of the diagnosis support AI. Then, the draft creation unit 103 outputs the draft of the medical certificate to the draft sending unit 104. Specifically, the draft creation unit 103 creates the draft of the medical certificate by refining a format of the text output by the diagnosis support AI. For example, in a case where the output of the diagnosis support AI is a bulleted list, the draft creation unit 103 creates the draft of the medical certificate by converting the bulleted list into sentences. Also, in a case where the diagnosis support AI outputs the certainty of the case as a score (numerical value), the draft creation unit 103 does not use a numerical value as is, but replaces the numerical value with words such as "highly likely" or "lowly likely" to create the draft of the medical certificate.

Note that in a case where the diagnosis support AI outputs a plurality of cases, the draft creation unit 103 can create a draft diagnosis for each case. At this time, the draft creation unit 103 may set priorities for the draft diagnoses according to the certainty of the cases. The draft creation unit 103 sets a higher priority for the draft diagnosis as the certainty of the case is higher.

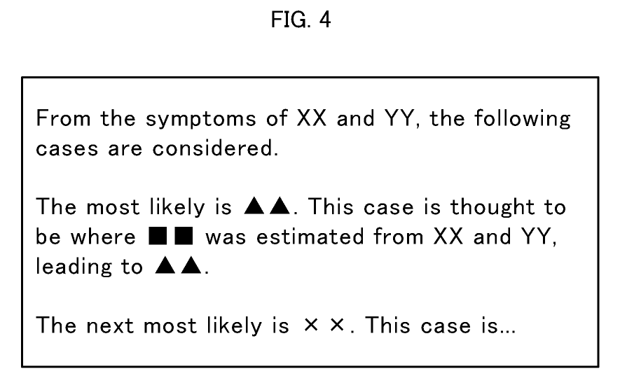

FIG. 4 is an example of a draft diagnosis. In the example of FIG. 4, the cases and the basis for selecting the cases are illustrated in descending order of certainty.

Returning to FIG. 3, the draft sending unit 104 refers to the cases included in the draft diagnosis, and selects the medical department to which the draft diagnosis will be sent. For example, the draft sending unit 104 can select a medical department from the draft diagnosis by a rule base. Such rules are, for example, rules that associate cases with medical departments. Also, the draft sending unit 104 may select a medical department using a machine learning model. This machine learning model is, for example, a trained machine learning model that has been trained to take a draft diagnosis as input and output a medical department.

The draft sending unit 104 sends the draft diagnosis to the terminal device 20 of the selected medical department. At this time, the draft sending unit 104 may notify the doctor who examined the patient (hereinafter also referred to as an "examining doctor") of the draft diagnosis and the medical department to which the draft diagnosis is to be sent, and obtain permission from the examining doctor to send the draft diagnosis.

Note that in a case where there are a plurality of drafts of medical certificates, the draft sending unit 104 may transmit all of them, or may transmit only those that satisfy predetermined conditions. Examples of predetermined conditions include: being a draft specified by the examining physician, being a draft with a priority higher than a predetermined rank, and being a draft which accuracy of the case is equal to or greater than a predetermined threshold.

Furthermore, in a case where there are the plurality of drafts of medical certificates and their cases belong to different medical fields, the draft sending unit 104 may classify the drafts by medical field and transmit the drafts to corresponding medical departments. Instead of the draft sending unit 104 performing the above classification, the draft creation unit 103 may classify the cases, and create respective drafts of medical certificates for each medical field.

In the above configuration, the diagnosis information input unit 101 is an example of a prompt creation means, the case candidate estimation unit 102 is an example of an estimation means, the draft creation unit 103 is an example of a draft creation means, and the draft sending unit 104 is an example of a selection means.

[Draft Creation Process]

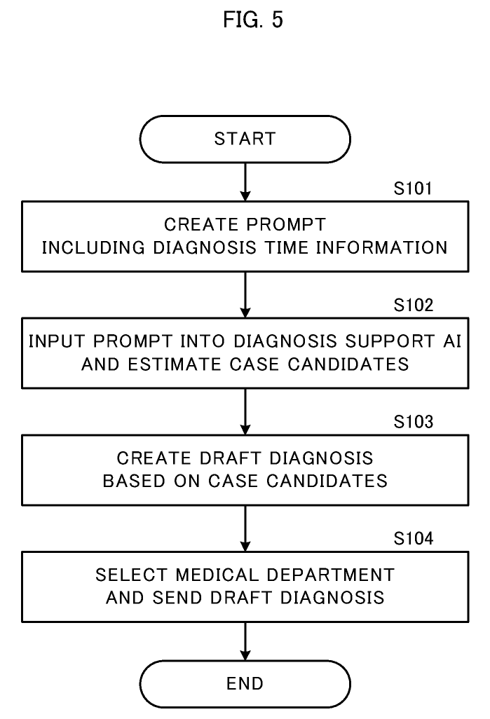

Next, the draft creation process for creating the draft of the medical certificate as described above will be explained. FIG. 5 is a flowchart of the draft creation process by the diagnosis support device 10. This draft creation is realized by the processor 12 illustrated in FIG. 2 executing a corresponding program prepared in advance and operating as each element illustrated in FIG. 3.

First, diagnostic information is input to the diagnosis support device 10 via the I/F 11. The diagnostic information is input to the diagnosis information input unit 101. The diagnosis information input unit 101 creates a prompt including the diagnostic information (step S101). The diagnosis information input unit 101 outputs the prompt to the case candidate estimation unit 102.

Next, the case candidate estimation unit 102 inputs the prompt into the diagnosis support AI, and estimates case candidates (step S102). The case candidate estimation unit 102 outputs the case candidates to the draft creation unit 103. Next, the draft creation unit 103 creates a draft of the medical certificate based on the case candidates (step S103). Specifically, the draft creation unit 103 creates the draft of the medical certificate by refining the format of the text output by the diagnosis support AI. The draft creation unit 103 outputs the draft of the medical certificate to the draft sending unit 104.

Next, the draft sending unit 104 selects a medical department to which the draft of the medical certificate will be sent, and sends the draft of the medical certificate to the selected medical department (step S104). After that, the draft creation process is terminated.

[Modifications]

Next, modifications of the first example embodiment will be described.

The diagnosis support device 10 may send the draft of the medical certificate to the medical department depending on how urgent the case is. For example, the draft sending unit 104 of the diagnosis support device 10 may send the draft of the medical certificate for cases with high urgency level to the medical department with higher priority than other drafts of medical certificates. In addition, the draft sending unit 104 may send an alert notification for the cases with high urgency level so as to be handled with higher priority than other patients. The cases with high urgency level include life-threatening cases such as those related to the brain or heart. Also, the urgency level may be expressed in three levels, such as "high," "medium," and "low," or two levels, such as "high" or "low."

The urgency level of each case can be estimated using diagnosis support AI. For example, the case candidate estimation unit 102 can estimate the urgency level of the case, in addition to the case and the basis for selecting that case, by inputting a prompt with added instructions for outputting the urgency level of the case into the diagnosis support AI.

Alternatively, the urgency level of the case may be estimated by the draft sending unit 104. The draft sending unit 104 can estimate the urgency level of the case and the medical department by the rule base. These rules are, for example, rules that associate cases with urgency and medical departments. The draft sending unit 104 may also use the machine learning model to estimate the urgency of the case and the medical department. This machine learning model is, for example, the trained machine learning model that has been trained to output the urgency level of the case and the medical department, with the draft of the medical certificate as input.

[Usage Example]

Next, a specific usage example of the diagnosis support system of the first example embodiment will be described. In this usage example, the draft of the medical certificate is used as a draft of a referral letter.

In a case where a patient says, "I visited Southeast Asia a week ago, and my stomach has been hurting ever since" during a consultation at an emergency hospital or a local office of the doctor, the terminal device 5 installed at the emergency hospital or the local office of the doctor sends that conversation as the diagnostic information to the diagnosis support device 10.

Based on the diagnostic information, the diagnosis support device 10 creates the draft of the referral letter concerning tropical region-related diseases. Then, the diagnosis support device 10 selects a medical department (a medical department knowledgeable concerning tropical region-related diseases) as a referral destination from the draft of the referral letter, and sends the draft of the referral letter. At this time, the diagnosis support device 10 may present the draft of the referral letter and the referral destination to the emergency hospital doctor or the local doctor, and obtain permission to send the draft of the referral letter. In this way, by using the diagnosis support system, the effort required to create the referral letter can be reduced, and the consultation at the medical department of the referral destination can proceed more smoothly.

<Second Example Embodiment>

Next, a second example embodiment will be described. The second example embodiment assumes a case where there is a group of medical institutions connected by the network, and there are the plurality of medical departments that can handle a certain case. The diagnosis support device according to the second example embodiment sends the draft of the medical certificate while considering the resources of each medical department. In this way, the diagnosis support device of the second example embodiment can level out the resources among the plurality of medical departments.

The diagnosis support device according to the second example embodiment has a similar system configuration and hardware configuration as the diagnosis support device 10 according to the first example embodiment, so their descriptions are omitted.

[Functional Configuration]

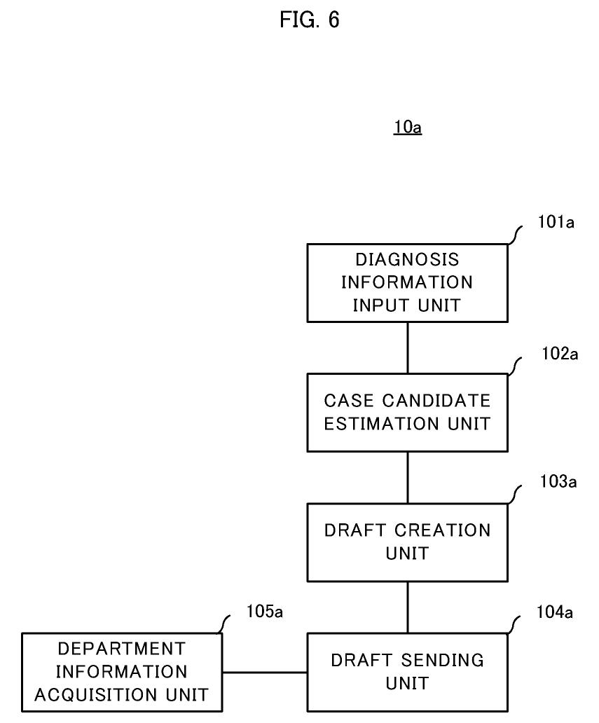

FIG. 6 is a block diagram illustrating a functional configuration of a diagnosis support device 10a according to the second example embodiment. The diagnosis support device 10a functionally includes a diagnosis information input unit 101a, a case candidate estimation unit 102a, a draft creation unit 103a, a draft sending unit 104a, and a department information acquisition unit 105a. Note that the diagnosis information input unit 101a, the case candidate estimation unit 102a, and the draft creation unit 103a have similar configurations respectively, and operate similarly to the diagnosis information input unit 101, the case candidate estimation unit 102, and the draft creation unit 103 of the diagnosis support device 10 according to the first example embodiment, so their descriptions are omitted.

To the diagnosis support device 10a, information related to each medical department (hereinafter also referred to as "medical department information") is input from the terminal device 20 of each medical department via the I/F 11. The medical department information is input to the department information acquisition unit 105a. The medical department information includes, for example, fixed information such as a location of the medical department and medical equipment (examination equipment) owned by the medical department, and variable information such as busyness of each doctor, schedules of doctors, and an operating status of medical equipment. The variable information is assumed to be periodically sent from the terminal device 20 of each medical department to the diagnosis support device 10a. The department information acquisition unit 105a outputs the medical department information to the draft sending unit 104a.

The draft sending unit 104a selects the medical department to which the draft of the medical certificate will be sent, based on the draft of the medical certificate and the medical department information.

Specifically, the draft sending unit 104a first selects one or more medical departments as candidates to which the draft will be sent, based on the draft of the medical certificate. The draft sending unit 104a can select one or more medical departments using the rule base or the machine learning model. The rule is, for example, a rule that associates each case with medical departments in a one-to-many relationship. The machine learning model is, for example, the trained machine learning model that has been trained to output one or more medical departments, with the draft of the medical certificate as input.

Next, the draft sending unit 104a refers to the medical department information, and selects an optimal medical department from among the plurality of medical departments. The draft sending unit 104a may select a medical department closest to a current location of patient as an optimal medical department. Also, the draft sending unit 104a may select a medical department where a doctor is not busy and where the doctor has availability in a schedule of that doctor as the optimal medical department. In addition to the schedule of each doctor, the draft sending unit 104a may select the optimal medical department taking into account availability of medical equipment necessary for examining and treating the case, and the reservation status of that medical equipment.

The draft sending unit 104a sends the draft of the medical certificate to the terminal device 20 of the selected medical department.

In the above configuration, the department information acquisition unit 105a is an example of a medical department information acquisition means.

[Draft Creation Process]

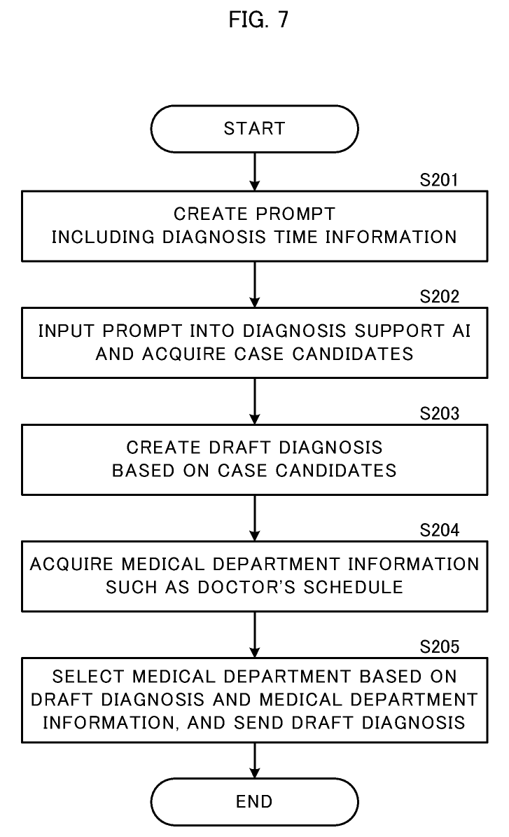

Next, a draft creation process for creating the draft of the medical certificate as described above will be explained. FIG. 7 is a flowchart of the draft creation process by the diagnosis support device 10a. This process is realized by the processor 12 illustrated in FIG. 2 executing a corresponding program prepared in advance and operating as each element illustrated in FIG. 6. Note that steps S201 to S203 are similar to steps S101 to S103 of the first example embodiment illustrated in FIG. 4, so their descriptions are omitted.

To the diagnosis support device 10a, medical department information is input from the terminal device 20 of each medical department via the I/F 11. The medical department information is input to the department information acquisition unit 105a (step S204). The department information acquisition unit 105a outputs the medical department information to the draft sending unit 104a.

Next, the draft sending unit 104 selects a medical department to which the draft of the medical certificate is sent, based on the draft of the medical certificate and the medical department information. Then, the draft sending unit 104 sends the draft of the medical certificate to the selected medical department (step S205). After that, the draft creation process is terminated.

[Modifications]

Next, modifications of the second example embodiment will be described. The following variations can be appropriately combined and applied to the second example embodiment.

(Modification 1)

The diagnosis support device 10a may send the draft of the medical certificate to the medical department taking into account the urgency level of the case. For example, the draft sending unit 104a of the diagnosis support device 10a may send the draft of the medical certificate for the urgent case to the medical department with higher priority than other drafts of medical certificates. In addition, the draft sending unit 104a may send an alert notification for the urgent cases to be handled with higher priority than other patients. The urgent cases include life-threatening cases such as those related to the brain or heart. Also, the urgency level may be expressed in three levels: "high," "medium," and "low," or two levels: "high" or "low."

The urgency level of the case can be estimated using diagnosis support AI. For example, the case candidate estimation unit 102a can estimate the urgency level of the case, in addition to the case and the basis for selecting that case, by inputting a prompt with an additional instruction for outputting the urgency level of the case into the diagnosis support AI.

Alternatively, the urgency level of the case may be estimated by the draft sending unit 104a. The draft sending unit 104a can estimate the urgency level of the case and the medical department by the rule base. The rules are, for example, rules that associate cases with urgency and the plurality of medical departments. Moreover, the draft sending unit 104a may use a machine learning model to estimate the urgency level of the case and the medical department. This machine learning model is, for example, a trained machine learning model that has been trained to output the urgency level of the case and one or more medical departments, with the draft of the medical certificate as input.

(Modification 2)

The diagnosis support device 10a may make the draft of the medical certificate available to each medical department, instead of sending the draft of the medical certificate. For example, the draft sending unit 104a of the diagnosis support device 10a publishes the draft of the medical certificate to the network of the medical institution group. At this time, the draft sending unit 104a may restrict access so that only the medical department selected by the draft sending unit 104a can access the draft of the medical certificate.

The person in charge of each medical department can access the diagnosis support device 10a and view the draft of the medical certificate. In addition, in a case where a medical department can handle the case, it may notify the diagnosis support device 10a. In a case where the diagnosis support device 10a receives the notification, it updates the published information. For example, the diagnosis support device 10a may add the name of the medical department corresponding to the draft of the medical certificate, or it may update the access restriction content so that only the corresponding medical department can view the draft of the medical certificate.

<Third Example Embodiment>

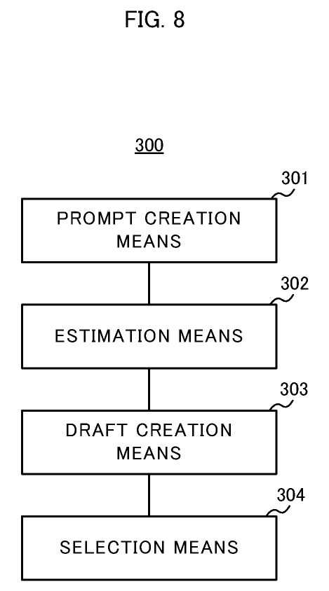

FIG. 8 is a block diagram illustrating a functional configuration of a diagnosis support device according to a third example embodiment. The diagnosis support device 300 functionally includes a prompt creation means 301, an estimation means 302, a draft creation means 303, and a selection means 304.

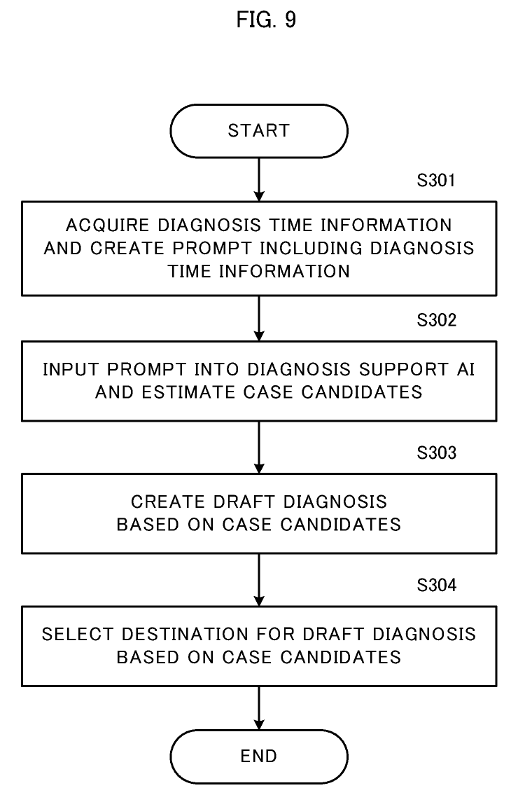

FIG. 9 is a flowchart of a process performed by the diagnosis support device according to the third example embodiment. The prompt creation means 301 acquires diagnosis information and creates a prompt including the diagnosis information (step S301). The estimation means 302 inputs the prompt into the diagnosis support AI and estimates case candidates (step S302). The draft creation means 303 creates the draft of the medical certificate based on the case candidates (step S303). The selection means 304 selects a destination for sending the draft of the medical certificate based on the case candidates (step S304).

According to the diagnosis support device 300 of the third example embodiment, it is possible to create the draft of the medical certificate and send the draft of the medical certificate to an appropriate department. This allows the diagnosis support device 300 to support decision-making by the doctor in the medical field.

A part or all of the example embodiments described above may also be described as the following supplementary notes, but not limited thereto.

(Supplementary note 1)

A diagnosis support device comprising:

a prompt creation means configured to acquire diagnosis information and creates a prompt including the diagnosis information;

an estimation means configured to input the prompt into a diagnosis support AI and estimates case candidates;

a draft creation means configured to create a draft of a medical certificate based on the case candidates; and

a selection means configured to select a destination for sending the draft of the medical certificate based on the case candidates.

(Supplementary note 2)

The diagnosis support device according to supplementary note 1, wherein the diagnosis support AI is created by additionally training a general-purpose generative AI using pairs of diagnosis information and information related to cases as training data.

(Supplementary note 3)

The diagnosis support device according to supplementary note 1, wherein

the estimation means estimates the case candidates, the basis for selecting the case candidates, and an accuracy indicating the possibility of the case candidates, and

the draft creation means creates the draft of the medical certificate including the case candidates and the basis in descending order of the accuracy.

(Supplementary note 4)

The diagnosis support device according to supplementary note 3, wherein the selection means sends the draft of the medical certificate for which the accuracy of the case candidates is equal to or higher than a predetermined threshold to the destination.

(Supplementary note 5)

The diagnosis support device according to supplementary note 1, wherein

the estimation means estimates an urgency level of the case candidates, and

the selection means sends the draft of the medical certificate and an alert notification to the destination in a case where the urgency level is high.

(Supplementary note 6)

The diagnosis support device according to supplementary note 1, further comprising a medical department information acquisition means configured to acquire medical department information from a plurality of medical departments, wherein

the selection means selects a medical department as the destination for sending the draft of the medical certificate based on the case candidates and the medical department information.

(Supplementary note 7)

The diagnosis support device according to supplementary note 6, wherein

the medical department information includes the degree of busyness of doctors and schedules of the doctors, and

the selection means selects a medical department as the destination for sending the draft of the medical certificate based on the degree of busyness of the doctors and the doctors' schedules.

(Supplementary note 8)

The diagnosis support device according to supplementary note 7, wherein

the medical department information includes the reservation status of examination equipment owned by the medical department, and

the selection means selects a medical department as the destination for sending the draft of the medical certificate based on availability of the examination equipment and its reservation status.

(Supplementary note 9)

A diagnosis support method executed by a computer, comprising:

acquiring diagnosis information and creating a prompt including the diagnosis information;

inputting the prompt into a diagnosis support AI and estimating case candidates;

creating a draft of a medical certificate based on the case candidates; and

selecting a destination for sending the draft of the medical certificate based on the case candidates.

(Supplementary note 10)

A program causing a computer to execute processing of:

acquiring diagnosis information and creating a prompt including the diagnosis information;

inputting the prompt into a diagnosis support AI and estimating case candidates;

creating a draft of a medical certificate based on the case candidates; and

selecting a destination for sending the draft of the medical certificate based on the case candidates.

While the present disclosure has been described with reference to the example embodiments and examples, the present disclosure is not limited to the above example embodiments and examples. Various changes which can be understood by those skilled in the art within the scope of the present disclosure can be made in the configuration and details of the present disclosure.

DESCRIPTION OF SYMBOLS

10, 10a Diagnosis support device

101, 101a Diagnosis information input unit

102, 102a Case candidate estimation unit

103, 103a Draft creation unit

104, 104a Draft sending unit

105a Department information acquisition unit

WHAT IS CLAIMED IS:

1. A diagnosis support device comprising:
at least one memory configured to store instructions; and
at least one processor configured to execute the instructions to:
acquire diagnosis information and creates a prompt including the diagnosis information;
input the prompt into a diagnosis support AI and estimates case candidates;
create a draft of a medical certificate based on the case candidates; and
select a destination for sending the draft of the medical certificate based on the case candidates.

2. The diagnosis support device according to claim 1, wherein the diagnosis support AI is created by additionally training a general-purpose generative AI using pairs of diagnosis information and information related to cases as training data.

3. The diagnosis support device according to claim 1, wherein the processor estimates the case candidates, the basis for selecting the case candidates, and an accuracy indicating the possibility of the case candidates, and the
processor creates the draft of the medical certificate including the case candidates and the basis in descending order of the accuracy.

4. The diagnosis support device according to claim 3, wherein the processor sends the draft of the medical certificate for which the accuracy of the case candidates is equal to or higher than a predetermined threshold to the destination.

5. The diagnosis support device according to claim 1, wherein
the processor estimates an urgency level of the case candidates, and
the processor sends the draft of the medical certificate and an alert notification to the destination in a case where the urgency level is high.

6. The diagnosis support device according to claim 1, wherein the processor is further configured to acquire medical department information from a plurality of medical departments, wherein
the processor selects a medical department as the destination for sending the draft of the medical certificate based on the case candidates and the medical department information.

7. The diagnosis support device according to claim 6, wherein
the medical department information includes the degree of busyness of doctors and schedules of the doctors, and
the processor selects a medical department as the destination for sending the draft of the medical certificate based on the degree of busyness of the doctors and the doctors' schedules.

8. The diagnosis support device according to claim 7, wherein the medical department information includes the reservation status of examination equipment owned by the medical department, and
the processor selects a medical department as the destination for sending the draft of the medical certificate based on availability of the examination equipment and its reservation status.

9. A diagnosis support method executed by a computer, comprising:
acquiring diagnosis information and creating a prompt including the diagnosis information;
inputting the prompt into a diagnosis support AI and estimating case candidates;
creating a draft of a medical certificate based on the case candidates; and
selecting a destination for sending the draft of the medical certificate based on the case candidates.

10. A non-transitory computer-readable recording medium storing a program causing a computer to execute processing of:
acquiring diagnosis information and creating a prompt including the diagnosis information;
inputting the prompt into a diagnosis support AI and estimating case candidates;
creating a draft of a medical certificate based on the case candidates; and
selecting a destination for sending the draft of the medical certificate based on the case candidates.
